(19) United States
(12) Patent Application Publication
(10) Pub. No.: US 2025/0111912 A1
(43) Pub. Date: Apr. 3, 2025

(54) INFORMATION PROCESSING SYSTEM, INFORMATION PROCESSING METHOD, AND NON-TRANSITORY COMPUTER-READABLE MEDIUM STORING PROGRAM

(71) Applicant: CureApp, Inc., Tokyo (JP)

(72) Inventors: Hikaru Takemura, Sunnyvale, CA (US); Shin Suzuki, Tokyo (JP)

(73) Assignee: CureApp, Inc., Tokyo (JP)

(21) Appl. No.: 18/816,562

(22) Filed: Aug. 27, 2024

Related U.S. Application Data
(60) Provisional application No. 63/541,108, filed on Sep. 28, 2023.

(51) Int. Cl. G16H 10/60

(52) U.S. Cl. CPC G16H 10/60

(57) ABSTRACT

A technology to generate a question sentence to acquire a message whose expression is customized in accordance with at least one of information related to age, gender, race or ethnicity, and region of a user from a machine learning system is provided.

An information processing system includes at least one processor, wherein the at least one processor accepts at least one of information related to age, gender, race or ethnicity, and region of a user who uses a lifestyle improvement support service, and generates a question sentence to instruct a machine learning model to make a change into an expression which is appropriate for a user layer identified by the accepted information, the machine learning model learning at least an expression that is in accordance with the information.

### CROSS REFERENCE TO RELATED APPLICATIONS

This application is based on and claims priority under 35 USC § 119 to U.S. provisional patent application Ser. No. 63/541,108, filed Sep. 28, 2023, the disclosure of which is incorporated herein by reference in its entirety.

### BACKGROUND

### Technical Field

The present disclosure relates to an information processing system, an information processing method, and a non-transitory computer-readable medium storing a program.

### Related Art

Currently, companies in the United States are increasingly interested in services which is to support prevention, diagnosis, and therapy of disease that attributes to lifestyle.

For establishing healthy lifestyle, it is important to provide appropriate information in accordance with a user's status, and support. Incidentally, in current services, a message is prepared for each information that is conveyed. However, even among same text, an impression that the user is given may be different for age or gender, and this difference of the impression may influence a motivation to improve lifestyle.

Aspects of the present disclosure provide a technology to generate a question sentence to acquire a message whose expression is customized in accordance with at least one of information related to age, gender, race or ethnicity, and region of the user from a machine learning system.

### SUMMARY

An aspect of the present disclosure provides an information processing system including at least one processor, wherein the at least one processor accepts at least one of information related to age, gender, race or ethnicity, and region of a user who uses a lifestyle improvement support service, and generates a question sentence to instruct a machine learning model to make a change into an expression which is appropriate for a user layer identified by the accepted information, the machine learning model learning at least an expression that is in accordance with the information.

Another aspect of the present disclosure provides an information processing method including: accepting at least one of information related to age, gender, race or ethnicity, and region of a user who uses a lifestyle improvement support service; and generating a question sentence to instruct a machine learning model to make a change into an expression which is appropriate for a user layer identified by the accepted information, the machine learning model learning at least an expression that is in accordance with the information.

Still another aspect of the present disclosure provides a non-transitory computer readable medium storing a program that causes a computer to execute an information processing method including: accepting at least one of information related to age, gender, race or ethnicity, and region of a user who uses a lifestyle improvement support service; and generating a question sentence to instruct a machine learning model to make a change into an expression which is appropriate for a user layer identified by the accepted information, the machine learning model learning at least an expression that is in accordance with the information.

According to the aspects of the present disclosure, it is possible to generate a question sentence to acquire message whose expression is customized in accordance with at least one of information related to age, gender, race or ethnicity, and region of the user from a machine learning system.

### BRIEF DESCRIPTION OF DRAWINGS

FIG. 1 illustrates a configuration example of an information processing system assumed in Exemplary embodiment 1;

FIG. 2 illustrates a hardware configuration of a support system;

FIG. 3 illustrates a hardware configuration of a user terminal;

FIG. 4 is a sequence diagram illustrating an overview of a support service provided through the information processing system;

FIG. 5 is a sequence diagram illustrating a procedure for creating message whose expression is customized for each user;

FIG. 6 shows an example of generating a question sentence by the question generating system;

FIG. 7 shows an example of outputting a reply sentence which a question answering system outputs;

FIG. 8 shows another example of outputting a reply sentence which a question answering system outputs;

FIG. 9 shows another example of outputting a reply sentence which a question answering system outputs;

FIG. 10 illustrates an example of transmitting customized message;

FIG. 11 shows another example of outputting a reply sentence which a question answering system outputs;

FIG. 12 shows another example of outputting a reply sentence which a question answering system outputs.

### DETAILED DESCRIPTION

Hereinafter, exemplary embodiments of the present disclosure will be described with reference to the drawings.

### Exemplary Embodiment 1

### <System Configuration>

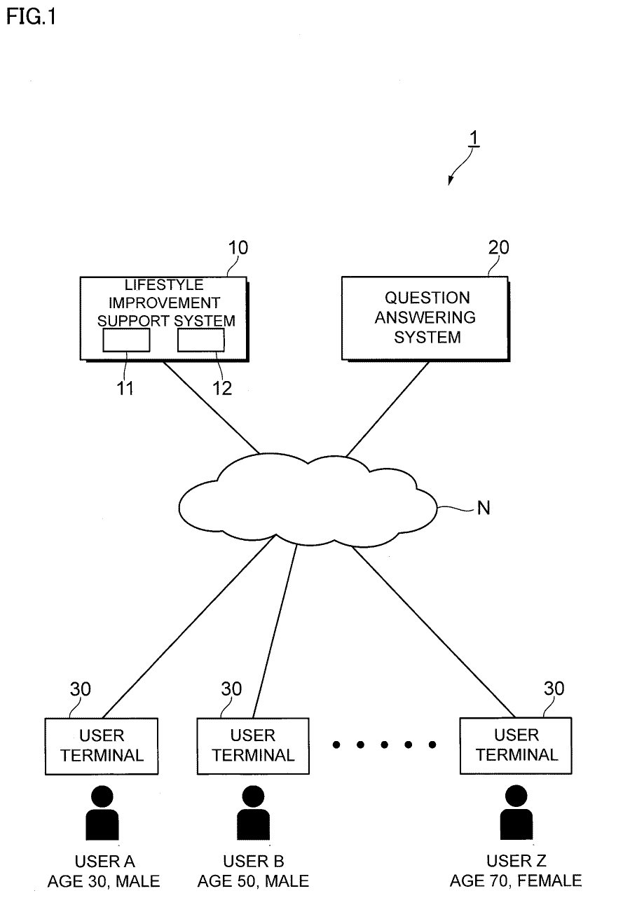

FIG. 1 illustrates a configuration example of an information processing system 1 assumed in Exemplary embodiment 1.

The information processing system 1 shown in FIG. 1 includes a lifestyle improvement support system (hereinafter referred to as “support system”) 10 that provides a lifestyle improvement support service (hereinafter referred to as “support service”), a question answering system 20 that accepts question in natural language and outputs an answer for the question, a user terminal 30 operated by a user of the support services, and a network N that connects these terminals.

The support system 10 in the exemplary embodiment is a system that distributes applications that support lifestyle improvement (hereinafter referred to as “support app”), stores user data entered through the support app, provides information to support lifestyle improvement, and so on.

The support system 10 in the exemplary embodiment is operated by a company or organization where a user works or the like. However, an enterprise or the like entrusted by the company or organization, and so on where the user works may operate the support system 10.

The support app in the exemplary embodiment is installed in the user terminal 30, and used for a record of daily life of the user. A record of daily life includes, for example, blood pressure values, weight, pulse, history of activity, and a record of feelings and moods.

The support system 10 in the exemplary embodiment includes a message generating system 11 and a question generating system 12, in relation to function providing information to support lifestyle improvement.

The message generating system 11 is a computer system that generates a message addressed to each user. The message generating system 11 in the exemplary embodiment is configured with a single server. However, the message generating system 11 may be configured with plural servers which collaborate mutually.

The message here includes, for example, an email addressed to an email address, a Social Networking Service (SNS) addressed to an account, and a Short Message Service (SMS) addressed to a cellphone number.

The message generating system 11 generates various kinds of messages (hereinafter referred to as “first message”) that contribute to improve lifestyle, based on the user data.

Note that, the message generating system 11 transmits a message (hereinafter referred to as “second message”) that an expression of the first message is changed in accordance with at least one of age, gender, race or ethnicity, and region of the user who is addressed, to a target user. The expression here includes, for example, a tone and manners.

In the exemplary embodiment, age, gender, race or ethnicity, and region are all registered in the support system 10 in advance. The region may be, for example, residence or workplace of the user, and may be identified by administrative division including these. Note that, the region may be a country, a locality or the like. By the way, the region may be registered not only one but also plurally. For example, both residence and workplace may be registered as the region.

The second message here is obtained by providing a question answering system 20 with the question sentence generated by the question generating system 12, and the first message.

The question generating system 12 is a computer system that generates the question sentence provided to the question answering system 20. The question generating system 12 in the exemplary embodiment is configured with a single server. However, the question generating system 12 may be configured with plural servers which collaborate mutually.

In the exemplary embodiment, the question generating system 12 generates a question sentence to instruct to change the message into a message whose expression is appropriate for a user layer identified by age, gender, race or ethnicity, and region of the user to whom the message is addressed. Note that, information related to age, gender, race or ethnicity, and region of the user is provided from the message generating system 11 to the question generating system 12, as the question sentence.

The question answering system 20 is a so-called computer system that provides natural language processing service. The question answering system 20 is not limited to a dedicated AI-type chatbot system, but may be a general-purpose AI-type chatbot system.

The question answering system 20 in the exemplary embodiment includes functions of; using a machine learning model learning an expression that is in accordance with age, gender, race or ethnicity, and region; changing an expression of the first message provided from the message generating system 11 to an expression in accordance with age, gender, race or ethnicity, and region of the user which are specified by the question sentence; and outputting the changed expression of the first message as a reply sentence.

However, the machine learning model does not need to learn expressions which are in accordance with all combinations among age, gender, race or ethnicity, and region.

In other words, the machine learning model may be a model learning an expression that is in accordance with any one of age, gender, race or ethnicity, and region, or these optional combinations. For example, the machine learning model may be a model learning an expression that is in accordance with the combination of the age and gender. In addition, for example, the machine learning model may be a model learning an expression that is in accordance with race or ethnicity. Moreover, for example, the machine learning model may be a model learning an expression that is in accordance with the region where the user lives or activates.

Note that, the machine learning model does not need to be a model learning only expression that is in accordance with any one of age, gender, race or ethnicity, and region, or these optional combinations. For example, the machine learning model may learn a variety of conversations or the like, in addition to the above-described expression.

The question sentence may be generated in accordance with the machine learning model which is used in generating message. This is because, for example, even if the machine learning model learning an expression that is in accordance with the combination of age and gender is instructed to make a change into an expression that is in accordance with race or ethnicity, desired result is not obtained.

However, in that case, plural machine learning models may be used properly, whose learned information is different. For example, when there are a machine learning model A, learning an expression that is in accordance with the combination of age and gender, and a machine learning model B, learning an expression that is in accordance with region, the first message and the question sentence (which instructs to make a change into an expression that is in accordance with the combination of age, gender and region) may be provided to the machine learning model A, and second message of a first version may be acquired, thereafter, the second message of the first version and the question sentence (which instructs to make a change into an expression that is in accordance with the combination of age, gender and region) may be provided to the machine learning model B, and the second message of a second version may be acquired.

Furthermore, two question sentences may be prepared in accordance with the plurality of machine learning models, and the second message may be generated to be target in accordance with each question sentence.

For example, when there are a machine learning model A, learning an expression that is in accordance with the combination of age and gender, and a machine learning model B, learning an expression that is in accordance with region, the first message and the question sentence (which instructs to make a change into an expression that is in accordance with age and gender) may be provided to the machine learning model A, and a second message of a first version may be acquired, thereafter, the second message of the first version and the question sentence (which instructs to make a change into an expression that is in accordance with region) may be provided to the machine learning model B, and the second message of a second version may be acquired.

The user terminal 30 is a computer terminal in which the support app is installed. The user terminal 30 in the exemplary embodiment includes, for example, a smart phone, a tablet-type computer, a laptop-type computer, and a desktop-type computer.

The user terminal 30 can communicate with the support system 10 through the network N. The user terminal 30 uploads the user data entered through the support app to the support system 10, and the support services are provided by the support system 10.

The network N is, for example, a local area network (LAN), the Internet, or a mobile communication system (4G, 5G).

### <Hardware Configuration of System>

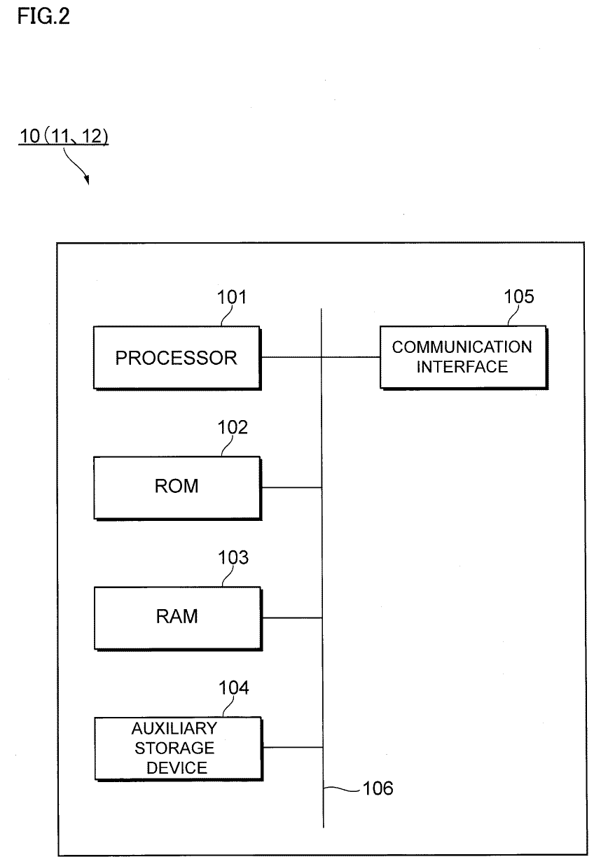

FIG. 2 illustrates a hardware configuration of the support system 10. Note that, the message generating system 11 and the question generating system 12 which constitute the support system 10 include a hardware configuration that is similar to the support system 10.

The support system 10 includes a processor 101, a Read Only Memory (ROM) 102 storing a Basic Input Output System (BIOS), etc., a Random Access Memory (RAM) 103 used as a work area of the processor 101, an auxiliary storage device 104, and a communication interface 105. Each device is connected via a bus and other signal wires 106.

The processor 101 is a device that implements various kinds of functions through the execution of programs. In the exemplary embodiment, the support app and Operating System (OS) are collectively called programs.

The processor 101, ROM 102, and RAM 103 function as a computer.

The auxiliary storage device 104 is configured with, for example, a hard disk device or a semiconductor storage.

The communication interface 105 is an interface for communicating with other servers or terminals via the network N. The communication interface 105 complies with Ethernet (registered trademark), Wi-Fi (registered trademark), mobile communication systems and other communication standards.

### <Data Stored in Auxiliary Storage Device>

### <Support System>

The auxiliary storage device 104 in the support system 10 stores the support app, and user data collected through the support app or the like.

The user data includes, for example, user attributes (gender, date of birth, age, height, weight), blood pressure values, activity records, mood records, physical condition records, medical examination history, operation history of the lifestyle improvement support application, biological characteristics, psychological characteristics, social characteristics, habits, goal achievement status, etc. However, the user data does not need to be all of such information, but may be part thereof.

The blood pressure values are, for example, systolic blood pressure (i.e., the highest blood pressure value) and diastolic blood pressure (i.e., the lowest blood pressure value).

The activity records include, for example, usage history of the support app, medications records, dietary records, smoking records, drinking records, and exercise records. The activity records are an example of information about activities.

The medications records include, for example, the substance and number of medications taken, and the time at which the medications were taken. The medications records are an example of information about medications.

The dietary records include, for example, the substance of the meals consumed, and the amount of salt taken. The dietary records are an example of information about diet.

The smoking records include, for example, the presence or absence of smoking, the date and time of smoking, and the number of cigarettes smoked. The smoking records are an example of information about smoking.

The drinking records include, for example, the date and time of drinking, the amount of alcohol intake, and the type of alcohol. The drinking records are an example of information about drinking.

The exercise records include, for example, records related to sports as well as records of activities in daily life such as walking, shopping, and cleaning. The exercise records are an example of information about exercise.

The mood records include, for example, a perceived mood. The mood records are an example of information about mood.

The physical condition records include, for example, a perceived physical condition or symptoms. The physical condition records are an example of information about physical condition.

The medical examination history includes, for example, the start date of the therapy, the date of the consultation, the details of the therapy, and advice from the doctor or other healthcare professionals. The medical examination history is an example of information about medical examination.

The operation history of the support app includes, for example, the operation history related to the activation operation and the entry operation of blood pressure values and reflection.

The biological characteristics include, for example, the presence or absence of other diseases, the presence or absence of injuries during therapy, the presence or absence of knee or foot pain, the presence or absence of experience in hypertensive therapy, the number of years since the high blood pressure was pointed out, the intensity of seasoning at home, and the amount of intake of food.

The psychological characteristics include, for example, expectations for the support app, willingness to acquire knowledge and information that contribute to improve lifestyle, feeling that salt reduction is challenging, feeling inability to alter the sense of taste, and a psychological resistance to leaving meals.

The social characteristics include, for example, wake time, bedtime, work pattern (such as shifts, day duty, and night duty), days of the week to work, the start time of the work, the time to return home, regular days off, and the presence or absence of a heater in a locker room.

The habits include, for example, an exercise habit, a weight measurement habit, a habit of calorie checking on food labels, a habit of low-fat food choices, a habit of not taking caffeine after 4 p.m., a habit of eating and drinking after 10 p.m., a habit of skipping breakfast, a habit of eating between meals, a habit of bathing one hour before bedtime, a habit of stretching and massaging before bedtime, and a habit of sleeping for more than six hours.

The goal achievement status includes, for example, the relationship between the user's status and the goal set by the doctor, etc. for each user.

Note that the above-described information can be classified into subjective information and objective information.

### <Message Generating System>

In the auxiliary storage device 104 of the message generating system 11, for example, a template of the first message sent to the user to support improvement of the lifestyle is stored. The template of the first message is prepared in accordance with the timing when a message is transmitted, a purpose to notify the message and a content that is notified. The template here includes, for example, a template used in a question for the user, a template used in an answer for the user and a template used in providing information and advice for the user.

### <Question Generating System>

In the auxiliary storage device 104 of the question generating system 12, for example, a template of a question sentence which is provided to the question answering system 20 is stored. In the case of the exemplary embodiment, a section of age and a section of gender in the template are blank.

### <Hardware Configuration of User Terminal>

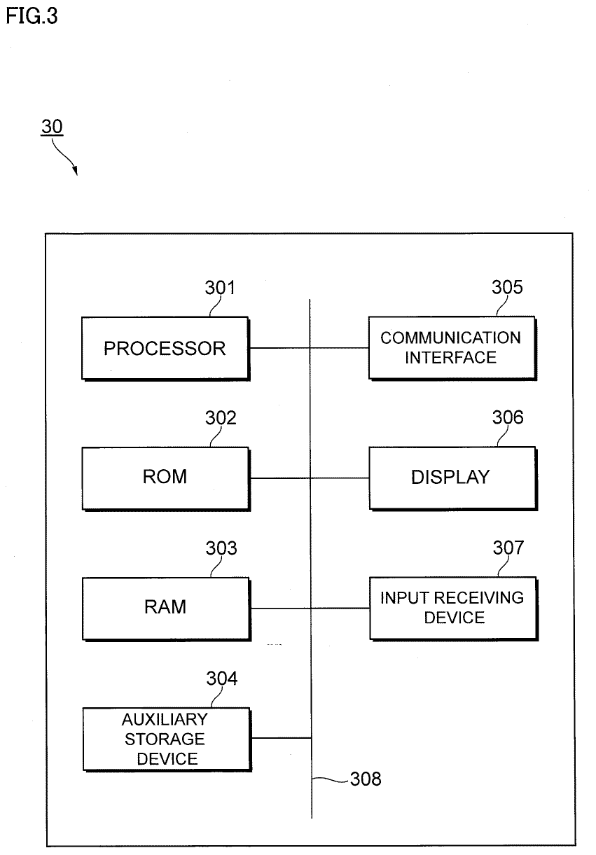

FIG. 3 illustrates a hardware configuration of the user terminal 30.

The user terminal 30 shown in FIG. 3 includes a processor 301, a ROM 302 storing a BIOS, etc., a RAM 303 used as a work area of the processor 301, an auxiliary storage device 304, a communication interface 305, a display 306, and an input receiving device 307. Each device is connected via a bus and other signal wires 308. Note that, the user terminal 30 may include a camera or a receiver of such as global positioning system (GPS) radio waves, or the like.

The communication interface 305 is an interface for communicating with other terminal devices or servers via the network N, or an interface for communicating with surrounding devices. The communication interface 305 complies with Ethernet (registered trademark), Wi-Fi (registered trademark), Bluetooth (registered trademark), Universal Serial Bus (USB), mobile communication systems and other communication standards.

The display 306 is, for example, a liquid crystal display or an organic Electro Luminescence (EL) display.

The input receiving device 307 is, for example, a mouse or a keyboard. By the way, in the case that the user terminal 30 is a laptop-type computer, a tablet-type computer or a smart phone, an electrostatic capacitance type touch sensor with permeability that does not interfere with the visibility of the image displayed on the display 306 may be used as the input receiving device 307. The device which is combined with this type touch sensor and display is called touch panel.

### <Processing Sequence Executed by Information Processing System>

### <Overview of Support Service>

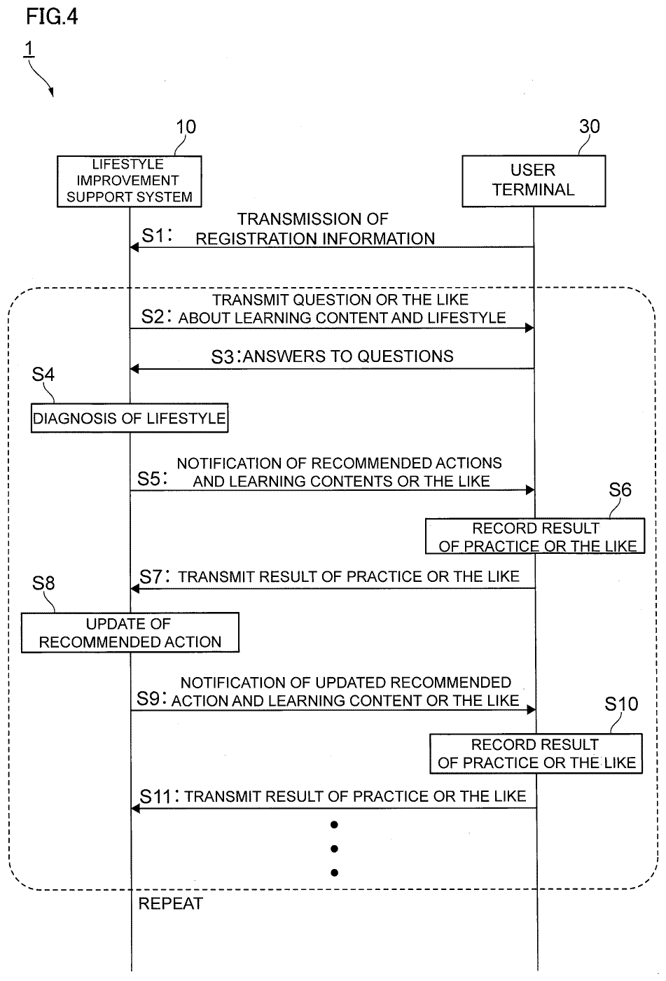

FIG. 4 is a sequence diagram illustrating an overview of a support service provided through the information processing system 1.

The processing sequence shown in FIG. 4 is implemented through the execution of programs by the processor 101 (refer to FIG. 2) which constitutes the support system 10, and the processor 301 (refer to FIG. 3) which constitutes the user terminal 30. The reference sign S shown in the figure represents a step.

### [Step 1]

In the case of FIG. 4, the user registration information is transmitted from the user terminal 30 to the support system 10. For example, the measurement values of blood pressure and pulse, information about medications taken or the like are transmitted as the registration information. In the case of the exemplary embodiment, the entry and transmission of the registration information is conducted through the support app.

### [Step 2]

The support system 10 transmits questions about learning contents and lifestyle problems to the user terminal 30.

The learning contents include knowledge and information that contribute to improve lifestyle. The learning contents are prepared for each disease, for example. In the case of the exemplary embodiment, high blood pressure is assumed as a disease. Note that the learning contents may be text-based, static-image-based, moving-image-based, or speech-based.

The questions related to the lifestyle are questions about the habits and preferences in daily life of each user. The questions in the exemplary embodiment are prepared to include, for example, six categories of diet, exercise, weight loss, sleep, stress, and alcohol.

### [Step 3]

The user terminal 30 transmits the answer to the questions by the user to the support system 10. Note that, the answer to the questions is, for example, entered through the selection of options by the user.

### [Step 4]

The support system 10 diagnoses the lifestyle of the target user based on the received answers. In the case of the exemplary embodiment, for example, score of the target user is calculated for each of the above-described six categories. In the case of the exemplary embodiment, the three categories of the low score are extracted as the featured categories which are likely to contribute to improve lifestyle. Note that, the extracted featured categories are notified as a result of diagnoses to the user.

### [Step 5]

The support system 10 notifies the user terminal 30 of the recommended actions and the learning content or the like related to the featured categories. The recommended action here is an action that contributes to improve the featured categories. However, it is not necessary to notify one recommended action per one featured category, and more than two recommended actions per one featured category may be notified.

In the case of the exemplary embodiment, the support system 10 determines the recommended action which is notified to the user, however, the mechanism that the user chooses the action to be practiced by his/herself may be adopted.

### [Step 6]

The user terminal 30 records the result of practice for the recommended action and the measurement result of blood pressure value or the like in the auxiliary storage device 304 (refer to FIG. 3). In the case of the exemplary embodiment, the result of practice for the recommended action and the measurement value are input through the support app. Note that, the result of practice and the measurement result or the like are recorded as user data in the auxiliary storage device 304.

### [Step 7]

The user terminal 30 transmits the recorded result of practice or the like to the support system 10. The transmission of the result of practice or the like may be performed on each input of the user data, and also may be performed at the timing of access to the support system 10 by the user.

### [Step 8]

The support system 10 updates the recommended actions periodically. In the case of the exemplary embodiment, the support system 10 changes at least part of the actions recommended for the user per one week. For example, two of the three recommended actions are replaced by new recommended actions. The new recommended actions are selected from among previously unrecommended actions as much as possible.

### [Step 9]

The support system 10 notifies the user terminal 30 of the updated recommended actions and leaning contents or the like.

### [Step 10]

The user terminal 30 records the result of practice for the recommended action and the measurement result of blood pressure value or the like in the auxiliary storage device 304 (refer to FIG. 3).

### [Step 11]

The user terminal 30 transmits the recorded result of practice or the like to the support system 10.

Hereinafter, the processing operation corresponding to Steps 8 to 11 is executed every week.

Note that, in the case of the exemplary embodiment, the processes from Step 2 are carried out repeatedly every month. In other words, the featured categories are reviewed and updated on a monthly basis, and the recommended actions to improve the updated featured categories are presented and put into practice repeatedly.

### <Customizing Message>

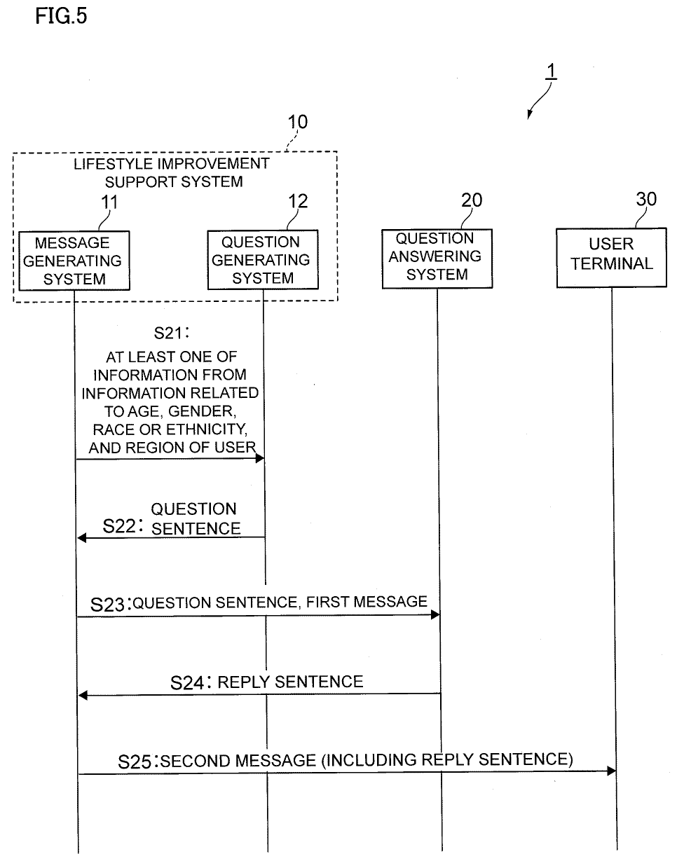

FIG. 5 is a sequence diagram illustrating a procedure for creating message whose expression is customized for each user. Note that, it is assumed that the question generating system 12 shown in FIG. 5 is able to generate the question sentence that is in accordance with at least one of information related to age, gender, race or ethnicity, and region, and the question answering system 20 is able to make a change into the expression that is in accordance with at least one of information related to age, gender, race or ethnicity, and region.

### [Step 21]

Firstly, the message generating system 11 provides the question generating system 12 with at least one of information related to age, gender, race or ethnicity, and region of a user who is the destination of the message.

### [Step 22]

For example, when information related to age and gender of the user is received, the question generating system 12 generates a question sentence to instruct to make a change into an expression which is appropriate for a user layer identified by the accepted information related to age and gender, and responds to the message generating system 11.

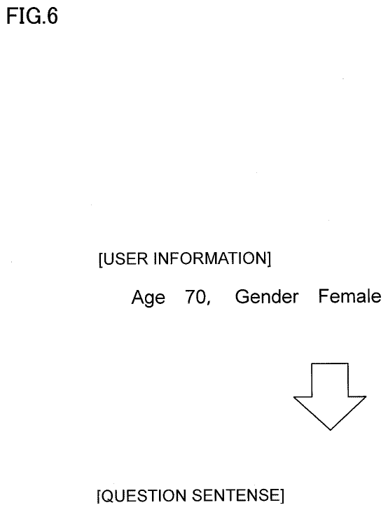

FIG. 6 shows an example of generating a question sentence by the question generating system 12.

The example shown in FIG. 6 is a case that the user information is provided as “Age: 70”, “Gender: Female”. In FIG. 6, as the question sentence, “You are an AI assistant that changes tone and voice in writing targeting demographics specified. Age: 70, Gender: Female” is generated.

### [Step 23]

The message generating system 11 that receives the question sentence transmits the question sentence and the first message to the question answering system 20.

The first message here is for example, the learning contents, questions about the lifestyle, or information that supports improvement of lifestyle. These are examples of information related to lifestyle.

### [Step 24]

The question answering system 20 generates a reply sentence that the expression of the first message is changed corresponding to the question sentence, and transmits the reply sentence to the message generating system 11.

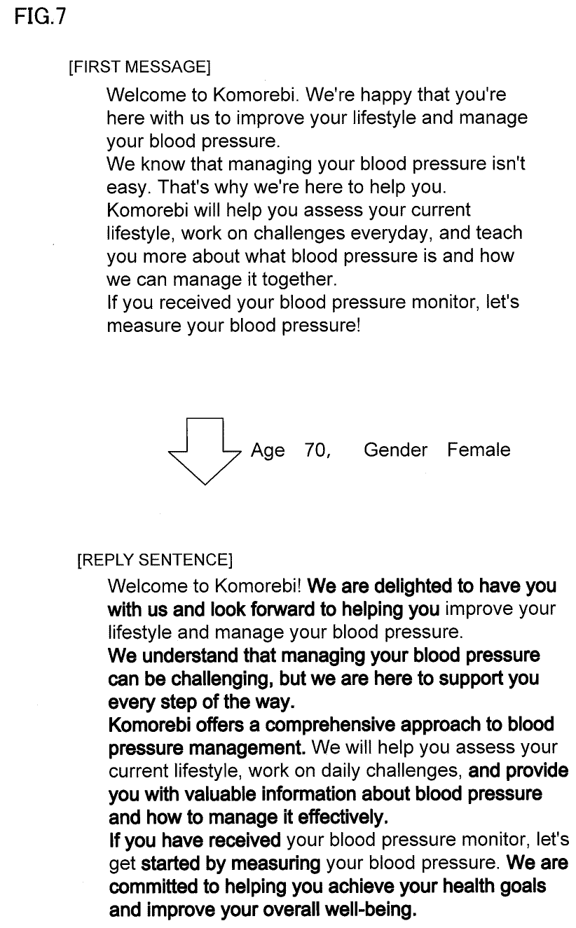

FIG. 7 shows an example of outputting a reply sentence which a question answering system 20 outputs. The reply sentence shown in FIG. 7 is a text that the expression of the first message is changed for a 70-year-old female. Note that, major change is shown in bold type.

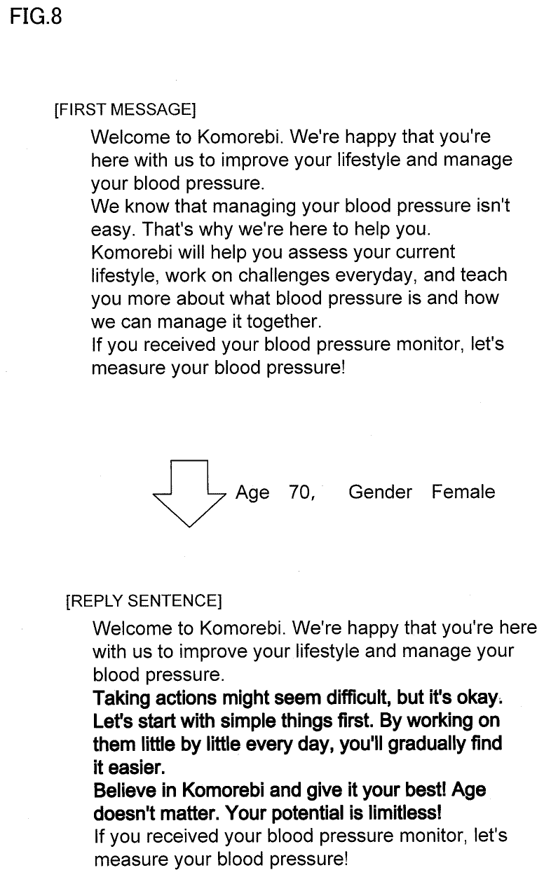

FIG. 8 shows another example of outputting a reply sentence which the question answering system 20 outputs. The reply sentence shown in FIG. 8 is also a text that the expression of the first message is changed for the 70-year-old female. Note that, the first message in FIG. 8 is a message that is shown when a diagnosis result of lifestyle is not good.

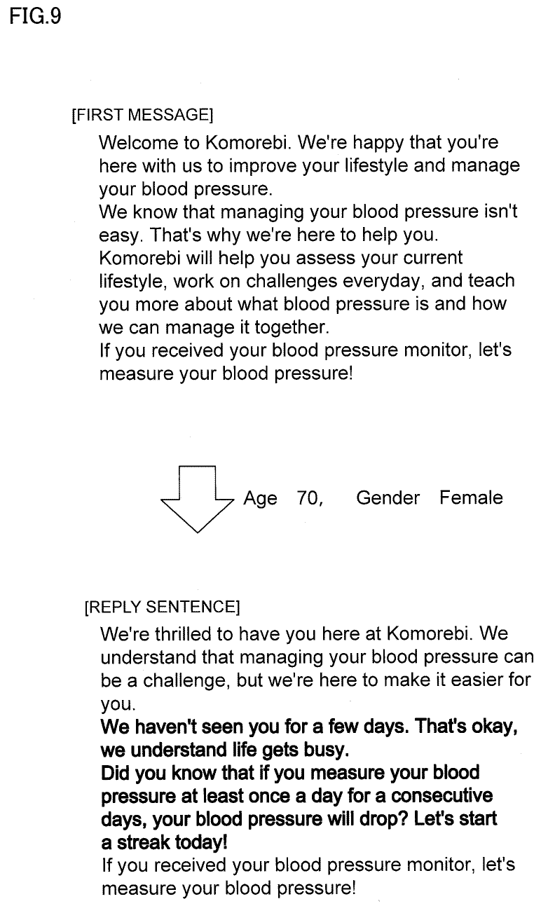

FIG. 9 shows another example of outputting a reply sentence which the question answering system 20 outputs. The reply sentence shown in FIG. 9 is also text that the expression of the first message is changed for the 70-year-old female. Note that, the first message in FIG. 9 is a message that is shown when a practicing status of the recommended action is not good.

### [Step 25]

The message generating system 11 which receives the reply sentence from the question answering system 20 transmits a second message whose main body is the reply sentence or a second message attached with the reply sentence to the user terminal 30. Thus, transmitting message whose expression is customized for each user who uses the lifestyle improvement support service is implemented.

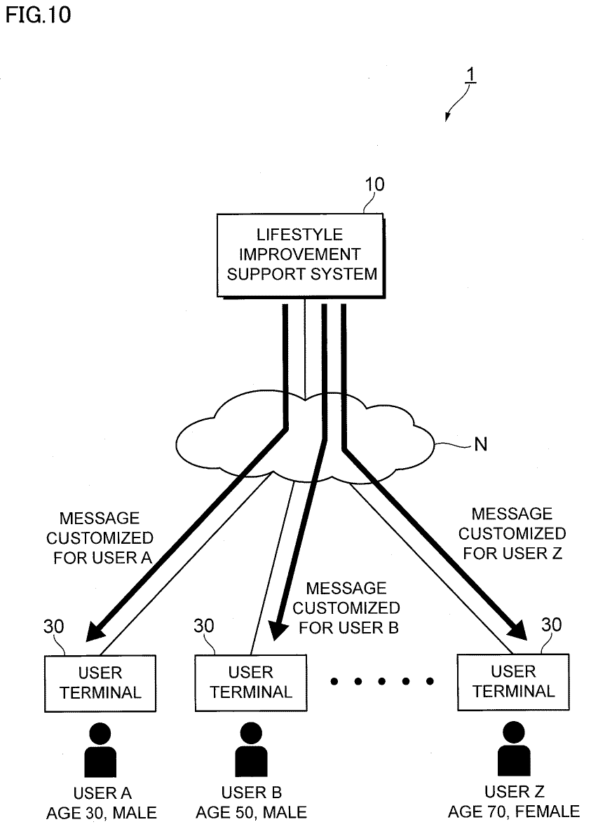

FIG. 10 illustrates an example of transmitting a customized message.

In FIG. 10, components corresponding to those of FIG. 1 are provided with same reference signs. In FIG. 10, the relationship between the lifestyle improvement support system 10 that is a transmitter of the message and the user terminal 30 is featured.

In case of FIG. 10, it is assumed that substance of the first message which is transmitted to the user A, user B and user Z is same. In other words, it is assumed that original message is common among the user A, user B and user Z.

However, the support system 10 in the exemplary embodiment transmits a message whose expression is customized for 30-year-old male to the user A, transmits a message whose expression is customized for 50-year-old male to the user B, and transmits a message whose expression is customized for 70-year-old female to the user Z.

### Other Examples of Generating the Question Sentence

In the above, a case is described, which is the case that the question answering system 20 (refer to FIG. 5) is instructed to make a change into the expression that is in accordance with age and gender, however, this is not limited to the above.

For example, in the Step 21, a case is also considered, which is the case that the information related to the race or ethnicity of the user is provided to the question generating system 12. In this case, a question sentence to instruct to make a change into an expression that is in accordance with race or ethnicity is generated in the Step 22, and responded to the message generating system 11. For example, as the question sentence, “You are an AI assistant that changes tone and voice in writing targeting demographics specified. Race/Ethnicity Japanese” is responded.

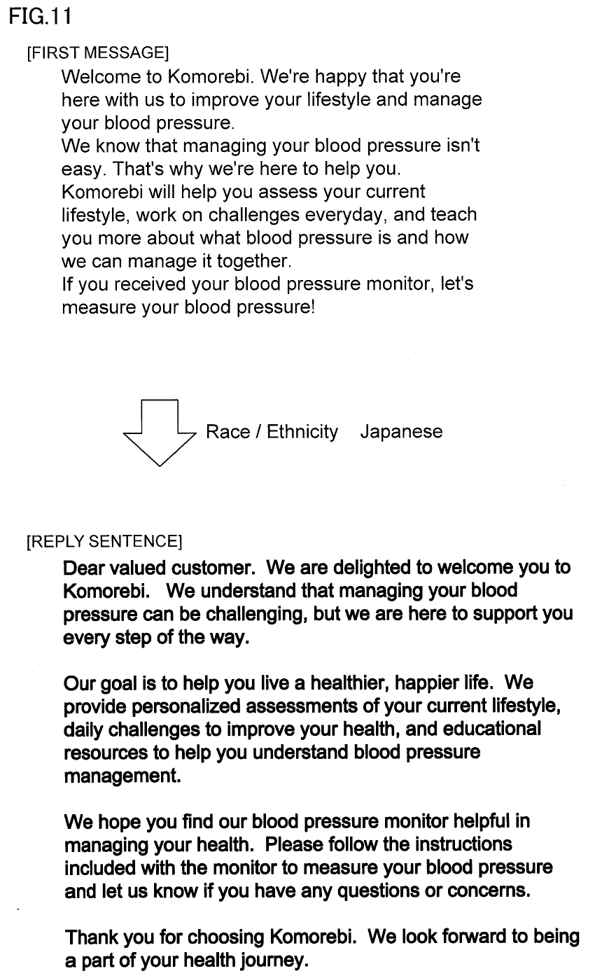

FIG. 11 shows another example of outputting a reply sentence which the question answering system 20 outputs. The reply sentence shown in FIG. 11 is a text that the expression of the first message is changed for Japanese.

In addition, for example, in the Step 21, a case is also considered, which is the case that the information related to registered region of the user is provided to the question generating system 12. In this case, a question sentence to instruct to make a change into an expression which is in accordance with region is generated in the Step 22, and responded to the message generating system 11. For example, as the question sentence, “You are an AI assistant that changes tone and voice in writing targeting demographics specified. Region with a lot of sunshine” is responded.

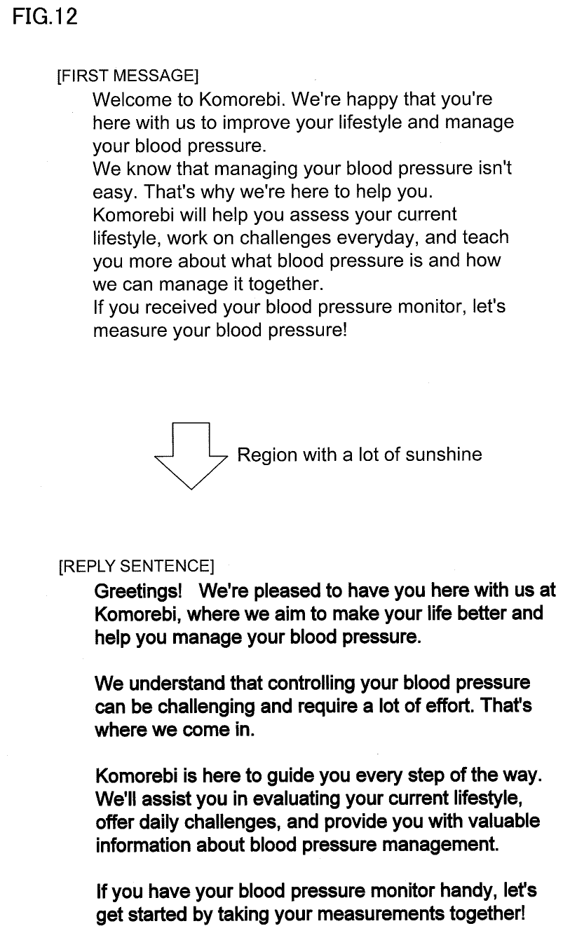

FIG. 12 shows another example of outputting a reply sentence which the question answering system 20 outputs. The reply sentence shown in FIG. 12 is a text that the expression of the first message is changed for the user who lives in the region with a lot of sunshine. In the reply sentence shown in FIG. 12, the text is made a change into the expression that is somewhat cheerful. However, the expression corresponding to the region with a lot of sunshine depends on teacher data or the like which the machine learning model uses for learning. Therefore, even if the substance of the question is same, when the machine learning model that is used is different, the expression is made a change into the different one.

### <Brief Conclusion>

According to the information processing system 1 (refer to FIG. 1) by the exemplary embodiment, it is possible to transmit a message whose expression is customized for each user.

As the result, receptivity or sympathy degree for the message increases, in comparison with a case that the message whose expression is uniform is transmitted, regardless of the user's age or gender, and it becomes to be easy to obtain effect of improving lifestyle.

### Other Exemplary Embodiments

(1) So far, the exemplary embodiments of the present disclosure have been described, but the technical scope of the present disclosure is not limited to the scope of the above-described exemplary embodiments. Various modifications or improvements added to the above-described exemplary embodiments may be apparently included in the technical scope of the present disclosure from the description of the claims.
(2) The processor in the above-described exemplary embodiments refers to a processor in a broad sense including, in addition to a general-purpose processor (for example, a Central Processing Unit (CPU)), a dedicated processor (for example, a Graphical Processing Unit (GPU), an Application specific Integrated Circuit (ASIC), a Field Programmable Gate Array (FPGA), a program logic device, etc.).

Moreover, the operation of the processor in each of the above-described exemplary embodiments is not limited to a single processor, but may be performed in cooperation by multiple processors. In addition, the order of execution of each operation in the processor is not limited to the order described in each of the above-described exemplary embodiments, but may be changed individually.
(3) In the above-described exemplary embodiments, it is assumed that the operator of the support system 10 (refer to FIG. 1) is a corporation or an enterprise or the like where a user works, however, the operator may also be an enterprise that operates a public medical insurance system or a private insurance system, or a medical profession that provides medical services.
(4) In the above-described exemplary embodiments, the corporation or the enterprise which operates the support system 10 operates the message generating system 11 and the question generating system 12, however, the operator of the message generating system 11 and the operator of the question generating system 12 may be different.
(5) In the above-described exemplary embodiments, the support system 10 includes the message generating system 11 and the question generating system 12, however, the support system 10 may be a system that the message generating system 11 and the question generating system 12 are integrated.
(6) The support services in the above-described exemplary embodiments aim to support the prevention, diagnosis, and therapy of high blood pressure, but can also be applied to other diseases.

The support services can be applied to customize messages in services that support the improvement of lifestyle related to, for example, nicotine addiction, insomnia disorder, depression, diabetes, alcoholism, and obesity.

By the way, in the case of nicotine addicted users, for example, carbon monoxide (CO) in exhaled breath, nicotine concentration in saliva, date and time of smoking, the number of cigarettes smoked or the number of times of smoking, user cognitive information (way of thinking, values), exercise (the number of steps, length of exercise time, travel distance), other diseases (diabetes), medications, blood pressure, heart rate, pulse, current physical condition (headache, irritation, feeling good, nausea, etc.), and all or part of the answers for assumed causes are stored as the user data. These information items are examples of information related to smoking.

In addition, in the case of insomnia users, all or part of, for example, bedtime, wake time, sleep hours, sleep efficiency, the number and substance of meals, exercise records, and medications records are stored as the user data. The bedtime, wake time, sleep hours, and sleep efficiency are examples of information related to sleep.

In addition, in the case of depressed users, all or part of, for example, mood records, action records, medications records, sleep records, and appetite records is stored as the user data.

In addition, in the case of users with diabetes, all or part of, for example, the number and substance of meals, exercise records, medications records, weight, blood pressure value, and blood sugar level is stored as the user data. The blood sugar level is an example of information about the blood sugar level.

In addition, in the case of alcoholic users, all or part of, for example, medications records, presence or absence of drinking, and substance of drinking is stored as the user data.

In addition, in the case of users with obesity, all or part of, for example, a target weight, therapy start date, the number of days of therapy, optimum intake calorie, physical condition history (mood), weight history, calorie intake history, calorie intake date and time, medications history (dose, medications date and time), therapy history (therapy, substance of advice, presence or absence of self-care by the user), medical care history, blood data (aspartate aminotransferase (AST), alanine aminotransferase (ALT)), and CT values is stored as the user data.
(7) In the above-described exemplary embodiments, the age of 70 is illustrated as an example of age that is provided in the Step 21, and cases which the ages are 30, 50 and 70 are illustrated in the Step 25, however, these examples do not mean that the user's age is specified in increments of 10, but intend that using actual age such as the age of 33 or 54. However, the user's age may be specified in increments of 5 or 10.

### CONCLUSION

The disclosure examples described in the above-described exemplary embodiments are shown below.

(((1)))

An information processing system includes at least one processor, wherein the at least one processor accepts at least one of information related to age, gender, race or ethnicity, and region of a user who uses a lifestyle improvement support service, and generates a question sentence to instruct a machine learning model to make a change into an expression which is appropriate for a user layer identified by the accepted information, the machine learning model learning at least an expression that is in accordance with the information.

According to the information processing system, it is possible to generate a question sentence to acquire message whose expression is customized in accordance with at least one of information related to age, gender, race or ethnicity, and region of the user from a machine learning system.

(((2)))

The information processing system described in (((1))), wherein the processor provides the machine learning model with the question sentence and a first message addressed to the user, and transmits a second message whose main body is a reply sentence from the machine learning model or a second message attached with the reply sentence to the user.

According to the information processing system, it is possible to transmit the message whose expression is customized in accordance with at least one of information related to age, gender, race or ethnicity, and region of the user.

(((3)))

The information processing system described in (((1))), wherein the processor provides the machine learning model with the question sentence and a first message that is in accordance with information related to the user's lifestyle, and transmits the second message whose main body is a reply sentence from the machine learning model or the second message attached with the reply sentence to the user.

According to the information processing system, it is possible to transmit the message whose expression is customized in accordance with at least one of information related to age, gender, race or ethnicity, and region of the user.

(((4)))

The information processing system described in (((3))), wherein the processor generates the first message in accordance with a diagnosis result of the user's lifestyle.

According to the information processing system, it is possible to transmit the message whose expression is customized in accordance with at least one of information related to age, gender, race or ethnicity, and region of the user.

(((5)))

The information processing system described in (((3))), wherein the processor generates the first message in accordance with the user's history of activity for the diagnosis result of the user's lifestyle.

According to the information processing system, it is possible to transmit the message whose expression is customized in accordance with at least one of information related to age, gender, race or ethnicity, and region of the user.

(((6)))

An information processing method including: accepting at least one of information related to age, gender, race or ethnicity, and region of the user who uses the lifestyle improvement support service; and generating a question sentence to instruct a machine learning model to make a change into an expression which is appropriate for a user layer identified by the accepted information, the machine learning model learning at least an expression that is in accordance with the information.

According to the information processing method, it is possible to generate a question sentence to acquire the message whose expression is customized in accordance with at least one of information related to age, gender, race or ethnicity, and region of the user from a machine learning system.

Note that, in this information processing method, it is possible to combine technologies corresponding to (((2))) to (((5))).

(((7)))

A non-transitory computer readable medium storing a program that causes a computer to execute an information processing method including: accepting at least one of information related to age, gender, race or ethnicity, and region of the user who uses a lifestyle improvement support service; and generating a question sentence to instruct a machine learning model to make a change into an expression which is appropriate for a user layer identified by the accepted information, the machine learning model learning at least an expression that is in accordance with the information.

According to the program, it is possible to generate a question sentence to acquire message whose expression is customized in accordance with at least one of information related to age, gender, race or ethnicity, and region of the user from a machine learning system.

Note that, in this information processing method, it is possible to combine technologies corresponding to (((2))) to (((5))). It is intended that the scope of the invention be defined by the following claims and their equivalents.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The exemplary embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing system comprising:
at least one processor, wherein
the at least one processor accepts at least one of information related to age, gender, race or ethnicity, and region of a user who uses a lifestyle improvement support service,
and generates a question sentence to instruct a machine learning model to make a change into an expression which is appropriate for a user layer identified by the accepted information, the machine learning model learning at least an expression that is in accordance with the information.

2. The information processing system according to claim 1, wherein the processor provides the machine learning model with the question sentence and a first message addressed to the user, and transmits a second message whose main body is a reply sentence from the machine learning model or a second message attached with the reply sentence to the user.

3. The information processing system according to claim 1, wherein the processor provides the machine learning model with the question sentence and a first message that is in accordance with information related to the user's lifestyle, and transmits the second message whose main body is the reply sentence from the machine learning model or the second message attached with the reply sentence to the user.

4. The information processing system according to claim 3, wherein the processor generates the first message in accordance with a diagnosis result of the user's lifestyle.

5. The information processing system according to claim 3, wherein the processor generates the first message in accordance with the user's history of activity for a diagnosis result of the user's lifestyle.

6. An information processing method comprising:
accepting at least one of information related to age, gender, race or ethnicity, and region of a user who uses a lifestyle improvement support service; and
generating a question sentence to instruct a machine learning model to make a change into an expression which is appropriate for a user layer identified by the accepted information, the machine learning model learning at least an expression that is in accordance with the information.

7. A non-transitory computer readable medium storing a program that causes a computer to execute an information processing method comprising:
accepting at least one of information related to age, gender, race or ethnicity, and region of a user who uses a lifestyle improvement support service; and
generating a question sentence to instruct a machine learning model to make a change into an expression which is appropriate for a user layer identified by the accepted information, the machine learning model learning at least an expression that is in accordance with the information.
